(19) United States
(12) Patent Application Publication
(10) Pub. No.: US 2022/0249374 A1
(43) Pub. Date: Aug. 11, 2022

(54) RADIOTHERAPEUTIC MICROSPHERES

(71) Applicants: William T. Phillips, San Antonio, TX (US); Ryan Bitar, San Antonio, TX (US)

(72) Inventors: William T. Phillips, San Antonio, TX (US); Ryan Bitar, San Antonio, TX (US)

(21) Appl. No.: 17/611,929

(22) PCT Filed: May 21, 2020
(86) PCT No.: PCT/US2020/033983

§ 371 Date: Nov. 17, 2021

Related U.S. Application Data
(60) Provisional application No. 62/851,915, filed on May 23, 2019.

(51) Int. Cl. A61K 9/127; A61K 9/16; A61K 47/02; A61K 41/00

(52) U.S. Cl. CPC A61K 9/127; A61K 9/1652; A61B 17/12186; A61K 41/0052; A61K 47/02

(57) ABSTRACT

Certain embodiments are directed to compositions comprising and method for producing alginate microspheres that contain liposomes encapsulating a variety of useful substances. Substances of note that can be encapsulated in liposomes and loaded alginate microspheres include radiotherapeutics (e.g., rhenium-188), radiolabels (e.g., technetium-99m), chemotherapeutics (doxorubicin), magnetic particles (e.g., 10 m iron nanoparticles), and radio-opaque material (e.g., iodine contrast).

### CROSS REFERENCE TO RELATED APPLICATIONS

This application claims priority to U.S. Provisional Application Ser. No. 62/851,915 filed May 23, 2019, which is incorporated by reference in its entirety.

### STATEMENT REGARDING FEDERALLY FUNDED RESEARCH

None.

### BACKGROUND

Hepatocellular Carcinoma (HCC) is the most common type of liver cancer. It is the sixth most common type of cancer and third most common cause of cancer mortality. HCC is particularly aggressive and has a poor survival rate (five-year survival of <5%) and therefore remains an important public health issue worldwide (GlobalData Intelligence Center—Pharma, URL pharma.globaldata.com/HomePage, 2019). HCC is most commonly found in liver exhibiting cirrhosis, or scarring of the liver, which can be caused by many factors including Hepatitis B infections, Hepatitis C infections, chronic alcohol abuse, and aflatoxins commonly found fungi that can grow on certain crops such as corn. HCC is also found to be more common in males by a 2.4:1 ratio compared to females (Balogh et al., J Hepatocell Carcinoma 3:41-53, 2016).

The primary means of treating HCC without cirrhosis is removing the tumor by surgery (resection). However a tumor may not be deemed resectable if the patient already has impaired liver function, the tumor has spread to multiple locations or is too large, or if too little of the patient's liver would remain after resection to allow for liver function post-surgery. For patients with cirrhosis, the best treatment is a liver transplant, however due to the shortage of donor organs; the wait time for patients who meet the criteria for transplant is over 2 years.

For unresectable HCC several other nonsurgical options are available that attempt to reduce the size or number of tumors to delay disease progression and to improve patient indicators to allow for resection. The most common procedure is transarterial chemoembolization, in which the one of the two main blood vessels, the hepatic artery, is blocked (embolized) to cut off the blood supply of the tumor. Prior to embolization, a chemotherapeutic agent is injected into the artery to deliver it preferentially to the tumor cells. This approach leaves the hepatic portal vein intact and is therefore thought to preserve the health of non-tumor liver cells that mainly depend on it for blood supply. Recently, the use of beads that release chemotherapeutic agents over time have been suggested to increase the effectiveness of these treatments.

Similarly, transarterial radioembolization uses the same types of particles to block the blood supply of the tumor; however, instead of chemotherapeutic agents, the particles rely on radiation given off by isotopes such as Yttrium-90 (Y-90) embedded in the particles (microspheres) that are delivered to the tumor. A variant on this procedure, known as percutaneous local ablation, follows the radioembolization with multiple days of direct injections of ethanol to the tumor.

Lastly, there is microwave ablation that uses electromagnetic waves with frequencies greater than 900 kHz to heat the tumor to a temperature higher than 100° C. This allows for a faster and more uniform ablation of the tumor, but studies have yet to show any statistical difference in efficiency compared to radioembolization.

The standard of care for patients with HCC considered too advanced for resection or localized ablation is systemic chemotherapy. The only treatment that has shown an improvement in mean survival of treatment groups is Bayer's Nexavar (sorafenib), which only prolongs survival by three months. Thus, there is a need for additional treatment options for HCC and other cancers.

### SUMMARY OF THE INVENTION

Certain embodiments are directed to compositions comprising and method for producing liposome containing alginate microspheres, optionally the liposomes encapsulate a variety of useful substances. Substances of note that can be encapsulated in liposomes and loaded into alginate microspheres include radiotherapeutics (e.g., rhenium-188), radiolabels (e.g., technetium-99m), chemotherapeutics (doxorubicin), magnetic particles (e.g., 10 μm iron nanoparticles), and radio-opaque material (e.g., iodine contrast). In certain aspects, rhenium-188 liposomes in alginate microspheres (Rhe-LAMs) can be used for treatment of liver tumors, specifically hepatocellular carcinoma (HCC). In a more particular aspect HCC treatment can be through radioembolization, where the microspheres block the blood supply to the tumor from the artery, while the rhenium-188 also delivers a high dose of radiation that is primarily targeted to the cancer cells.

Microparticles produced by standard production methods frequently have a wide particle size distribution, lack uniformity, fail to provide adequate release kinetics or other properties, and are difficult and expensive to produce. In addition, the microparticles may be large and tend to form aggregates, requiring a size selection process to remove particles considered to be too large for administration to patients by injection or inhalation. This requires sieving and results in product loss. Certain embodiments described herein use an ultrasonic nozzle or nebulizer to produce liposome containing microspheres. An ultrasonic nebulizer uses high-frequency electrical energy to create vibrational, mechanical energy, typically employing a piezoelectric transducer. This energy is transmitted to the liquid or formulation to form microspheres either directly or through a coupling fluid, creating an aerosol containing microspheres, which are subsequently cured or cross-linked. Typically, ultrasonic energy disrupts the association or lipids forming a liposome. The results described herein are surprising and unexpected in that the liposomes resist the disruptive effects of ultrasound remaining intact during production processes resulting in the formation of smaller liposome containing alginate microspheres.

In certain aspects, liposome containing alginate microspheres (LAMs) are produce by spraying a liposome/alginate solution (liquid or feed source) into a curing solution having an alginate cross-linker. Typically, a liquid is supplied by powered pumps to simple or complex orifice nozzles that atomize the liquid stream into spray droplets that are cross-linked when exposed to the curing solution. Nozzles are often selected primarily on the desired range of flow rates needed and secondarily on the range of liquid droplet size. Any spray atomizer that can produce droplets from the liquids described herein can be used. Suitable spray atomizers include two-fluid nozzles, single fluid nozzles, ultrasonic nozzles such as the Sono-Tek™ ultrasonic nozzle, rotary atomizers or vibrating orifice aerosol generators (VOAG), and the like. In certain aspects, the nozzle is an ultrasonic nozzle, a 1 Hz to about 100 kHz nozzle. In one particular aspect the nozzle is a 25 kHz nozzle. In certain aspects, the spray atomizer can have one or more of the following specifications. (a) a 25 kHz to 180 kHz nozzle, in particular a 25 kHz nozzle. (b) a 1 to 10 W generator, in particular a 5.0 W generator. (c) a pump capable of a flow rate of 0.1 to 1.0 ml/min, in particular 0.5 ml/min (microbore may be necessary for a flow rate this low). The curing solution can be positioned to receive the atomized liquid. The distance between the nozzle and the curing solution can be varied between 1 to 10 cm, in particular 4 cm. the system can be activated for the entirety of nozzle usage. The generator can be activated and the pump can form liposome containing alginate microspheres (LAMs). Microspheres can be incubated at room temp (e.g., 20 to 30° C.) in the curing solution (e.g., CaCl₂) solution) for 1 to 10 minutes, in particular 5 minutes. In certain aspects, the microspheres can be spun down, for example at 1000-1200 rpm. Followed by abstracting the supernatant to wash the spheres of free reagents, e.g., unbound Re-188/Tc-99m. Microsphere solution can be passed through a 100 μm-pore stainless steel mesh for exclusion of any clumping that may have occurred during the cross-linking or centrifugation. These LAMs can be used for intraarterial administration. In certain aspects, the microspheres can be visualized under light microsopy, and dosimeter can be used to measure radioactivity retention in those LAMs loaded with radioactive materials.

Certain embodiments are directed to LAMs having a diameter of 1, 10, 20, 30, 40, 50, 60, 70, 80, 90, 100, 150, 200, 300, 350, 400, 450, to 500 μm, including all values and ranges there between (in certain aspects any of the values or subranges can be specifically excluded). In certain aspects, the LAMs have an average diameter of 20 to 80 μm, including all values and ranges there between. In certain aspects the ratio of liposome to alginate (w/w or v/v) is 4:1, 3:1, 2:1, 1:1, 1:2, 1:3, 1:4, including all ratios and ranges there between (in certain aspects any of the values or subranges can be specifically excluded). In certain aspects, the LAM comprises 10 to 80 weight percent liposome/lipid, 10 to 80 weight percent alginate solution, 0.01 to 5 weight percent alginate cross-linker, and 1 to 30 weight percent therapeutic and/or imaging agent.

As used herein, a “liposome” refers to a vesicle consisting of an aqueous core enclosed by one or more phospholipid layers. Liposomes may be unilamellar, composed of a single bilayer, or they may be multilamellar, composed of two or more concentric bilayers. Liposomes range from small unilamellar vesicles (SUVs) to larger multilamellar vesicles. LMVs form spontaneously upon hydration with agitation of dry lipid films/cakes which are generally formed by dissolving a lipid in an organic solvent, coating a vessel wall with the solution and evaporating the solvent. Energy is then applied to convert the LMVs to SUVs, LUVs, etc. The energy can be in the form of, without limitation, sonication, high pressure, elevated temperatures and extrusion to provide smaller single and multi-lamellar vesicles. During this process some of the aqueous medium is entrapped in the vesicle. Liposomes can also be prepared using emulsion templating. Emulsion templating comprises, in brief, the preparation of a water-in-oil emulsion stabilized by a lipid, layering of the emulsion onto an aqueous phase, centrifugation of the water/oil droplets into the water phase and removal of the oil phase to give a dispersion of unilamellar liposomes. Liposomes prepared by any method, not merely those described above, may be used in the compositions and methods of this invention. Any of the preceding techniques as well as any others known in the art or as may become known in the future may be used as compositions of therapeutic agents in or on a delivery interface of this invention. Liposomes comprising phospholipids and/or sphingolipids may be used to deliver hydrophilic (water-soluble) or precipitated therapeutic compounds encapsulated within the inner liposomal volume and/or to deliver hydrophobic therapeutic agents dispersed within the hydrophobic bilayer membrane. In certain aspects the liposome comprises lipids selected from sphingolipids, ether lipids, sterols, phospholipids, phosphoglycerides, and glycolipids. In certain aspects, the lipid includes, for example, DSPC (1,2-distearoyl-sn-glycero-3-phosphocholine).

As used herein, “alginate” refers to a linear polysaccharide that can be derived from seaweed. The most common source of alginate is the species Macrocystis pyrifera. Alginate is composed of repeating units of D-mannuronic (M) and L-guluronic acid (G), presented in both alternating blocks and alternating individual residues. Soluble alginate may be in the form of monovalent salts including, without limitation, sodium alginate, potassium alginate and ammonium alginate. In certain aspects, the alginate includes, but is not limited to one or more of sodium alginate, potassium alginate, calcium alginate, magnesium alginate, ammonium alginate, and triethanolamine alginate. Alginates are present in the formula in amounts ranging from 5 to 80% by weight, preferably in amounts ranging from 20 to 60% by weight, and most preferably about 50% by weight. In certain aspects, the alginate is ultra-pure alginate (e.g., Novamatrix ultra-pure alginate). Alginate can be cross-linked using ionic gelation provided through multivalent cations in solution, e.g., an aqueous or alcoholic solution with multivalent cations therein, reacting with alginates. Multivalent cations (e.g., divalent cations, monovalent cations are not sufficient for cross-linking alginate) for use with alginates include, but are not limited to calcium, strontium, barium, iron, silver, aluminum, magnesium, manganese, copper, and zinc, including salts thereof. In certain aspects, the cation is calcium and is provided in the form of an aqueous calcium chloride solution.

In certain aspects the therapeutic or imaging agent is a chemotherapeutic, radiotherapeutic, thermotherapeutic, or a contrast agent.

In certain aspects, a radiotherapeutic agent includes a radiolabel such as a beta emitter (¹³¹I, ⁹⁰Y, ¹⁷⁷Lu, ¹⁸⁶Re, ¹⁸⁸Re, any one of which can be specifically excluded) or gamma emitter (¹²⁵I, ¹²³I) In certain aspects, the radiotherapeutic agent is ¹⁸⁸Re. Furthermore, the term “radiotherapeutic” may be taken to more broadly encompass any radioactively-labeled moiety, and may include any liposome or LAM associated with or comprising a radionuclide. The liposome or LAM may be associated with a radionuclide through a chelator, direct chemical bonding, or some other means such as a linker protein.

In certain aspects, a chemotherapeutic agent includes, but is not limited to a chemical compound that inhibits or kills growing cells and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alcaloids, in particular vinblastine, vincristine, vindesine, vinflunine, and vinorelbine. Liposome can be used to carry hydrophilic agents as micelles can be used to carry lipophilic agents.

In general, the thermotherapeutic agents include a plurality of magnetic nanoparticles, or “susceptors,” of an energy susceptive material that are capable of generating heat via magnetic hysteresis losses in the presence of an energy source, such as, an alternating magnetic field (AMF). The methods described herein, generally, include the steps of administering an effective amount of a thermotherapeutic compound to a subject in need of therapy and applying energy to the subject. The application of energy may cause inductive heating of the magnetic nanoparticles which in turn heats the tissue to which the thermotherapeutic compounds were administered sufficiently to ablate tissue. In certain aspects, a thermotherapeutic agent includes, but is not limited to magnetite (Fe₃O₄), maghemite (γ-Fe₂O₃) and FeCo/SiO₂, and in some embodiments, may include aggregates of superparamagnetic grains of, for example, Co₃₆C₆₅, Bi₃Fe₅O₁₂, BaFe₁₂O₁₉, NiFe, CoNiFe, Co—Fe₃O₄, and FePt—Ag, where the state of the aggregate may induce magnetic blocking. In thermotherapy, the response of MNPs to AC magnetic field causes thermal energy to be dissipated into the surroundings, killing the tumor cells. Additionally, hyperthermia can enhance radiation and chemotherapy treatment of cancer. The term “hyperthermia”, as used herein, refers to heating of tissue to temperatures between about 40° C. and about 60° C. The term “alternating magnetic field” or “AMF”, as used herein, refers to a magnetic field that changes the direction of its field vector periodically, typically in a sinusoidal, triangular, rectangular or similar shape pattern, with a frequency of in the range of from about 80 kHz to about 800 kHz. The AMF may also be added to a static magnetic field, such that only the AMF component of the resulting magnetic field vector changes direction. It will be appreciated that an alternating magnetic field may be accompanied by an alternating electric field and may be electromagnetic in nature. In certain embodiments, the thermotherapeutic agent can be incorporated into alginate microspheres in the absence of lipids and as such form a thermotherpeutic containing alginate microsphere where the agent is not incorporated in a liposome but is incorporated in the alginate microsphere.

In certain aspects, a contrast or imaging agent includes, but is not limited a transition metal, carbon nanomaterials such as carbon nanotubes, fullerene and graphene, near-infrared (NIR) dyes such as indocyanine green (ICG), and gold nanoparticles. Transition metal refers to a metal in Group 3 to 12 of the Periodic Table of Elements, such as titanium (Ti), vanadium (V), niobium (Nb), tantalum (Ta), chromium (Cr), molybdenum (Mo), tungsten (W), manganese (Mn), iron (Fe), ruthenium (Ru), osmium (Os), iridium (Ir), nickel (Ni), copper (Cu), technetium (Tc), rhenium (Re), cobalt (Co), rhodium (Rh), iridium (Ir), palladium (Pd), platinum (Pt), silver (Ag), gold (Au), a lanthanide such as europium (Eu), gadolinium (Gd), lanthanum (La), ytterbium (Yb), and erbium (Er), or a post-transition metal such as gallium (Ga), and indium (In). In one aspect, the imaging modality is selected from the group comprising, Positron Emission Tomography (PET), Single Photon Emission Tomography (SPECT), Computed Tomography (CT), Magnetic Resonance Imaging (MRI), Ultrasound Imaging (US), and Optical Imaging. In another aspect of the invention, the imaging modality is Positron Emission Tomography (PET). The imaging agent includes, but is not limited to a radiolabel, a fluorophore, a fluorochrome, an optical reporter, a magnetic reporter, an X-ray reporter, an ultrasound imaging reporter or a nanoparticle reporter. In another aspect of the invention, the imaging agent is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium. In another aspect, the radiolabel is selected from the group comprising zirconium-89 (⁸⁹Zr), iodine-124 (¹²⁴I), iodine-131 (¹³¹I), iodine-125 (¹²⁵I) iodine-123 (¹²³I), bismuth-212 (²¹²Bi), bismuth-213 (²¹³Bi), astatine-221 (²¹¹At), copper-67 (⁶⁷Cu), copper-64 (⁶⁴Cu), rhenium-186 (¹⁸⁶Re), rhenium-186 (¹⁸⁸Re), phosphorus-32 (³²P), samarium-153 (¹⁵³Sm), lutetium-177 (¹¹⁷Lu), technetium-99m (^{99m}Tc), gallium-67 (⁶⁷Ga), indium-111 (¹¹¹In), thallium-201 (²⁰¹Tl) carbon-11, nitrogen-13 (¹³N), oxygen-15 (¹⁵O), fluorine-18 (¹⁸F), and rubidium-82 (⁸²Ru).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Image of two rabbits after intra-arterial injection into the hepatic artery, demonstrating embolic efficacy in the liver.

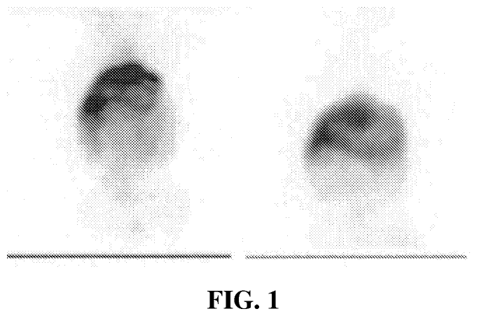

### DETAILED DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Embodiments are directed to therapeutic and/or diagnostic alginate microspheres, Certain aspects are directed to therapeutic alginate microspheres for intra-arterial embolic therapy. In a further aspect the therapeutic alginate microspheres are radiotherapeutic alginate microspheres. In certain embodiments ultrasonic spray atomization can be used to produce alginate microspheres. Methods described herein can be used to manufacture small (20-80 micron) homogeneous liposome containing alginate microspheres (LAMs). Larger rhenium liposomes encapsulated in microspheres of 250 microns in size have been described; however, smaller microspheres are needed for intra-arterial delivery, for example to hepatocellular carcinomas (HCCs) and other cancers. Certain aspects include:

Method of loading LAMs with a variety of anti-cancer drugs (example drug doxorubicin) using ultrasonic atomization that are held stably inside the Dox-LAMs with potential for slow release after intra-arterial delivery into a tumor.

Method of stably loading rhenium-188, Tc-99m or a variety of anti-cancer drugs into pre-formed LAMs. Surprisingly, the labeling agents or drugs are able to penetrate into the alginate microspheres and then enter into the liposomes where they become stably trapped.

Method of making magnetic alginate microspheres (MAMs) containing small 10 nanometer iron particles. The surprising discovery is that these small iron nanoparticles were stably retained inside of the alginate microspheres. The iron nanoparticles used for this discovered are currently under development for treatment of human prostate cancer in San Antonio via thermal heating in an alternating current field.

In certain aspects, Re-188 beta-emitting microsphere can be used for the treatment of liver cancer. This embolic, yet ultimately biodegradable, microcapsule can carry the inexpensive beta-emitting radionuclide Re-188. This therapeutic agent can be manufactured and administrated within a just few hours and permit high quality imaging. The proposed model involves encapsulating Re-188 liposomes into alginate microspheres.

This microsphere system is flexible as it can carry drugs in addition of radionuclides. For instance, in prior research, radiolabel liposomal doxorubicin was used with the radionuclide rhenium. This liposomal doxorubicin could potentially be incorporated into the microspheres for intra-arterial treatment of liver cancer. These dual modality microspheres could have improved therapeutic benefit. It may also be possible to incorporate radio-opaque material, iodine contrast, into the microspheres to assist in visualization of the tumor treatment during intra-arterial infusion.

### I. Alginate Microspheres

Alginate is a polysaccharide which forms a hardened gel matrix in the presence of divalent cations such as calcium and barium. Microspheres constructed from alginate have been investigated for the delayed release of therapeutic agents from the alginate matrix. Specifically, low molecular weight molecules (such as doxorubicin) can escape from the spheres and to the target tissue. Free radionuclides would be no exception and would most likely leak into systemic circulation if administered intraarterially. Thus, this invention is dependent upon the encapsulation of Re 188 in alginate microspheres, without permitting the radionuclide to escape the porous alginate interface. This disclosure proposes to successfully encapsulate Re-188 in microspheres by making alginate microspheres with Re labeled liposomes. The liposomes do not permit Re-188 to pass through the lipid bilayer and the liposomes are >100 nm, preventing them from being able to escape the porous interface of the alginate. These spheres are intended for direct intra-arterial delivery to liver tumors for radioembolization, thus a size range which can enter the capillary bed but not pass through (into systemic circulation) is required. Thus the proposed model is a means of producing alginate microspheres (20-80 μm) which contain Rhenium liposomes. As mentioned earlier, Tc-99m may substitute as the radionuclide in the place of Re-188 as the two radionuclides share similar chemistry. The radiolabeling procedure is practically synonymous.

Liposome formation. Construct ammonium sulfate gradient liposomes. Add phospholipids and cholesterol to a round-bottomed flask in appropriate amounts. Add chloroform or chloroform-methanol depending on lipid composition to dissolve lipids and form lipid solution. Conduct rotary evaporation on lipid solution to remove solvent and form lipid thin film. Temperature and evaporation time will vary based on lipid formulation. Desiccate lipid thin film under vacuum for at least 4 h. In certain aspects desiccation can be overnight. Rehydrate lipid thin film (e.g., 300 mM sucrose in sterile water) for injection at a predetermined total lipid concentration (e.g., 60 mM). Vortex solution and heat above lipid phase transition temperature until all lipids are in solution. Freeze lipid solution and lyophilize forming a dry powder. The dry powder is rehydrated in an appropriate buffer (e.g., ammonium sulfate in sterile water) to an appropriate total lipid concentration (e.g., 60 mM) forming a new solution. Vortex the solution vigorously and heat above lipid phase transition temperature until all lipids are in solution. Freeze the lipid solution with liquid nitrogen and then thaw in water bath set to temperature above the lipid phase transition temperature. Repeat freeze-thaw procedure for at least three cycles. Extrude liposome sample until desired particle diameter is achieved. After extrusion, final liposome product should be stored at 4° C. until needed. The liposomes can be characterized by laser light scattering particle sizing, pyrogenicity, sterility, and lipid concentration.

Alginate preparation. An alginate solution (e.g., 1, 2, 3, 4, 5, 6% w/v) is prepared in water or another appropriate buffer (e.g., HEPES buffer). The alginate solution is allowed to rest for at least 48 hrs to homogenize and eliminate air bubbles.

Cross-linking preparation. The cross-linking solution of 0.136 M CaCl-2H₂O and 0.05% w/v Tween 80 is prepared. In certain instances BaCl₂ is also an acceptable cross-linking agent.

Radiolabeled liposome preparation. Prepare a Sephadex G-25 column with buffer at pH 7.4. Typically, 1 column can be used for every 2 ml of liposomes. Drain buffer from the Sephadex G25 column reservoir and add liposomes onto the top of the column and elute with pH 7.4 buffer. To maximize yield and minimize dilution use the centrifugation method (rather than the gravity method) for desalting the liposomes before radiolabeling. To maximize yield and minimize efficiency, do not run the labeled liposomes through a Sephadex column. Washing the spheres in future steps will remove any free Re-188/Tc-99m.

Liposome/alginate solution preparation. Vortex liposome solution with alginate solution 1:1 by volume until homogenous.

Nozzle apparatus and use thereof. In certain aspects a nozzle apparatus is employed. The nozzle apparatus can have one or more of the following specifications. (a) For the purpose of intraarterial embolism, a 25 kHz nozzle is recommended. (b) Generator at 5.0 W. (c) Syringe pump at 0.5 ml/min (microbore may be necessary for a flow rate this low). (d) Place the crosslinking solution on stir plate and underneath nozzle (e.g., about 4 cm below). Activate for the entirety of nozzle usage. (e) Activate the generator and then activate the syringe pump forming liposome containing alginate microspheres. Let microspheres incubate at room temp in the CaCl₂) solution for 5 minutes. Spin down microspheres at 1000-1200 rpm and abstract the supernatant to wash the spheres of free Re-188/Tc-99m. It is recommended to wash the spheres by additionally re-suspending the pellet with sterile DI water. Centrifuge that mixture and abstract the supernatant. Resuspend washed spheres in sterile saline. Run the sphere/saline solution through a 100 μm-pore stainless steel mesh for exclusion of any clumping that may have occurred during the cross-linking or centrifugation. Draw up liposome containing microspheres in syringe for intraarterial administration.

It is anticipated that these microspheres will have the following significant advantages as compared to current Y-90 microspheres for the treatment of liver tumor by interventional radiology: Re-188 can be readily available and significantly less expensive than Y-90 microspheres. This is because a rhenium-188 generator can now be purchased on a one-time basis for a relatively low cost for a 500 mCi generator (enough to treat several patients a day for 4 months) or a 3,000 mCi generator (enough to treat 5-10 patients a day for 4 months). These generators can be used for up to 6 months by milking the Re-188 from a generator every day for 6 months. This generator can provide rapid manufacturing of Re-188 microspheres for dosing on short notice which could provide significant benefit to the patient considering the growth rate of liver tumors. The low cost and ready availability of Re-188 microspheres can provide a significant benefit in comparison with Y-90 microspheres which is manufactured in a reactor and requires a 2 weeks advanced order. Low cost and portability of the rhenium generator also may mean this technology could be easily made available in developing countries which have a higher incidence of liver tumors than the US.

Like Y-90, Re-188 has a high energy beta particle with a mean tissue path length of 4 mm in tissue. This tissue path length is important for intra-arterial therapy to provide an extensive micro field of radiation within the liver tumor. This beta energy and path length in tissue is twice as great as Re-186 currently used to treat glioblastoma. Unlike, Y-90, Re-188 has a 15% gamma photon in the ideal photon energy range for acquisition of very high-quality SPECT images for monitoring distribution and retention. In contrast, Y-90 does not emit a gamma photon and produces only Bremsstrahlung radiation with a photon flux at least 100-fold less than rhenium-188. Rhenium can be readily obtained from a Re-188 generator that can be located near the site of use of the rhenium-188 microspheres. This generator can last for 6 months and can provide rhenium-188 for treatments of thousands of patients at a relatively low cost.

In certain embodiments the microspheres can be produced via spray atomization. Conventional methods for atomization include air pressure and electrospraying. In certain aspects, the method uses ultrasonication as the method for producing microspheres with a tight size-range. Sono-tek Corp in Poughkeepsie, N.Y. constructs nozzles with an ultrasonicating atomizing surface which can rapidly atomize fluids with a narrow size range in comparison to conventional methods. Mean microsphere size is mainly dependent upon which frequency nozzle is selected for sphere production. Studies with the nozzle have found that spheres with a size range of 20-80 (mean of 44 microns) can be produced with a 25 kHz nozzle at a rate of 0.5 ml/min.

Alginate microspheres may also be manufactured using Microfluidization technology. Sizes of alginate microspheres that can be produced can range from 20-500 depending on the microfluidics system utilized. Alginate microspheres of 40 microns±3 microns can be prepared using microfluidization. This method has yet to be tested with radionuclides due to the time factor that this method introduces. Crosslinking via ultrasonication atomization takes minutes while construction of spheres with a single microfluidics chip may take a full day. Much radioactivity will have undergo decay before patient administration. Therefore, this method could be considered with either (A) the simultaneous utilization of many chips or (B) the utilization of a singular chip with multiple inlets/outlets.

It is contemplated that a significant benefit of using biodegradable alginate microspheres that contain liposomal nanoparticles is the potential to take advantage of the ingestion of liposome microspheres by intratumoral macrophages to improve the intratumoral distribution of the therapeutic agents within the tumor. It is further contemplated that this improved biodistribution would be due to phagocytosis of the degraded microsphere by macrophages that can move freely within the tumor. Macrophages have also been proposed as a mechanism to enhance tumor coverage of another type of nanoparticle with evidence showing nanoparticle movement from an injection site at a small region of the tumor to cover the whole tumor. Macrophage enhanced intratumoral coverage enhancement following intra-arterial delivery can include the degradable microsphere containing beta-emitting radionuclide nanoparticles have embolized an artery feeding the tumor. Macrophages can partially degrade the microsphere and ingested the nanoparticles and moved therapeutic radiation through portions of the tumor. The microsphere can be complete degraded, and macrophages have covered the tumor, including the invasive margins of the tumor.

A recent study has shown that when alginate microspheres of 250 microns in size are injected into the liver, a significant portion of these alginate microspheres degrade and spread within the tumor by 2 weeks. It is likely that using microspheres smaller than 100 μm will likely improve their biodegradability by macrophages as opposed to microspheres of >200 microns in size. Another approach to increase the degradation rate if needed would be to include other components, such as gelatin and glucomannan in the alginate microsphere. In prior research performed as part of a drug delivery grant from the Gates Foundation, we have shown that alginate microcapsules containing a significant portion of gelatin (collagen) (1:2 ratio of gelatin to alginate) and or glucomannan (1:2 ratio of glucomannan to alginate) also can still form stable alginate-based microspheres and can be stably radiolabeled with Tc-99m or Re-186. Changing the composition of the microsphere could potentially cause a more rapid macrophage degradation due to presence of collagenase in macrophages or increases M2 macrophage stimulation of mannose receptors on macrophages by glucomannan resulting in a more rapid phagocytosis and degradation of the hybrid alginate/glucomannan microspheres. Prior studies have shown that glucomannan can enhance macrophage uptake of nanoparticles. Ability to create degradable microspheres and control their time of degradation after administration could provide a significant advantage for this alginate-based manufacture of microspheres as compared to embolization with non-biodegradable glass or resin microspheres. Biodegradable microspheres may cause less damage to normal liver tissue than permanent glass or resin microspheres.

The Rhenium-microspheres can be used for the treatment of cancer by intra-arterial delivery with the initial cancer candidate treatment being liver cancer. This strategy can be extended to potentially to lung cancer. The availability of a low-cost rhenium-188 generator and alginate microsphere production make this therapy an inexpensive option for the treatment of cancer.

Microspheres containing Tc-99m liposomes (Tec-LAMs) which are a highly representative surrogate for rhenium-188 have been injected intra-arterially into the hepatic artery of rabbits and have demonstrated embolic efficacy in the liver as indicated by this image of 2 rabbits at 1 hour post-administration. After 24 hours there was minimal change in the images and both rabbits had a very similar appearance of the liver with very good retention. Note that there is no activity visualized in the lungs or in the kidney. The lack of visualization of activity in the lungs is very promising for these the Tec-LAMs. The currently clinically available microspheres containing Y-90 generally have 5 percent activity in the lungs which can be a limiting factor for therapy when shunting to the lungs is too high. The fact that no lung activity or renal activity is visualized is very encouraging and shows that the LAMs are embolic intra-arterially in the location in which they are injected and they do not fall apart in the circulation to any large degree over time. The development of Re-186 microspheres has been developed but has yet to be tested in vitro.

### II. Liposomes

Selection of the appropriate lipids for liposome composition is governed by the factors of: (1) liposome stability, (2) phase transition temperature, (3) charge, (4) non-toxicity to mammalian systems, (5) encapsulation efficiency, (6) lipid mixture characteristics, and the like. The vesicle-forming lipids preferably have two hydrocarbon chains, typically acyl chains, and a head group, either polar or nonpolar. The hydrocarbon chains may be saturated or have varying degrees of unsaturation. There are a variety of synthetic vesicle-forming lipids and naturally-occurring vesicle-forming lipids, including the sphingolipids, ether lipids, sterols, phospholipids, phosphoglycerides, and glycolipids (e.g., cerebrosides and gangliosides).

Phosphoglycerides include phospholipids such as phosphatidylcholine, phosphatidylethanolamine, phosphatidic acid, phosphatidylinositol, phosphatidylserine phosphatidylglycerol and diphosphatidylglycerol (cardiolipin), where the two hydrocarbon chains are typically between about 14-22 carbon atoms in length, and have varying degrees of unsaturation. As used herein, the abbreviation “PC” stands for phosphatidylcholine, and “PS” stand for phosphatidylserine. Lipids containing either saturated and unsaturated fatty acids are widely available to those of skill in the art. Additionally, the two hydrocarbon chains of the lipid may be symmetrical or asymmetrical. The above-described lipids and phospholipids whose acyl chains have varying lengths and degrees of saturation can be obtained commercially or prepared according to published methods.

Phosphatidylcholines include, but are not limited to dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoylphosphatidylcholine, stearoyl-palmitoylphosphatidylcholine, stearoyl-oleoyl-phosphatidylcholine, stearoyl-linoleoylphosphatidylcholine and palmitoyl-linoleoylphosphatidylcholine. As symetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. As used herein, the abbreviation “PC” refers to phosphatidylcholine. The phosphatidylcholine 1,2-dimyristoyl-sn-glycero-3-phosphocholine is abbreviated herein as “DMPC.” The phosphatidylcholine 1,2-dioleoyl-sn-glycero-3-phosphocholine is abbreviated herein as “DOPC.” The phosphatidylcholine 1,2-dipalmitoyl-sn-glycero-3-phosphocholine is abbreviated herein as “DPPC.”

In general, saturated acyl groups found in various lipids include groups having the trivial names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heneicosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and asymmetric phosphatidylcholines include myristoleoyl, palmitoleyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cis-octadecatrienoic, 11-cis-eicosenoic and 5-cis-8-cis-11-cis-14-cis-eicosatetraenoic.

Phosphatidylethanolamines include, but are not limited to dimyristoyl-phosphatidylethanolamine, dipalmitoyl-phosphatidylethanolamine, distearoyl-phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoethanolamine, depending on whether they are symmetric or assymetric lipids.

Phosphatidic acids include, but are not limited to dimyristoyl phosphatidic acid, dipalmitoyl phosphatidic acid and dioleoyl phosphatidic acid. Phosphatidic acids may also be referred to under IUPAC naming systems as 1,2-diacyl-sn-glycero-3-phosphate or 1-acyl-2-acyl-sn-glycero-3-phosphate, depending on whether they are symmetric or assymetric lipids.

Phosphatidylserines include, but are not limited to dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Phosphatidylserines may also be referred to under IUPAC naming systems as 1,2-diacyl-sn-glycero-3-[phospho-L-serine] or 1-acyl-2-acyl-sn-glycero-3-[phospho-L-serine], depending on whether they are symmetric or assymetric lipids. As used herein, the abbreviation “PS” refers to phosphatidylserine.

Phosphatidylglycerols include, but are not limited to dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Phosphatidylglycerols may also be referred to under IUPAC naming systems as 1,2-diacyl-sn-glycero-3-[phospho-rac-(1-glycerol)] or 1-acyl-2-acyl-sn-glycero-3-[phospho-rac-(1-glycerol)], depending on whether they are symmetric or assymetric lipids. The phosphatidylglycerol 1,2-dimyristoyl-sn-glycero-3-[phospho-rac-(1-glycerol)] is abbreviated herein as “DMPG”. The phosphatidylglycerol 1,2-dipalmitoyl-sn-glycero-3-(phospho-rac-1-glycerol) (sodium salt) is abbreviated herein as “DPPG”.

Suitable sphingomyelins include, but are not limited to brain sphingomyelin, egg sphingomyelin, dipalmitoyl sphingomyelin, and distearoyl sphingomyelin.

Other suitable lipids include glycolipids, sphingolipids, ether lipids, glycolipids such as the cerebrosides and gangliosides, and sterols, such as cholesterol or ergosterol. As used herein, the term cholesterol is sometimes abbreviated as “Chol.” Additional lipids suitable for use in liposomes are known to persons of skill in the art.

In certain aspects the overall surface charge of the liposome can be varied. In certain embodiments anionic phospholipids such as phosphatidylserine, phosphatidylinositol, phosphatidic acid, and cardiolipin are used. Neutral lipids such as dioleoylphosphatidyl ethanolamine (DOPE) may be used. Cationic lipids may be used for alteration of liposomal charge, as a minor component of the lipid composition or as a major or sole component. Suitable cationic lipids typically have a lipophilic moiety, such as a sterol, an acyl or diacyl chain, and where the lipid has an overall net positive charge. Preferably, the head group of the lipid carries the positive charge.

One of skill in the art will select vesicle-forming lipids that achieve a specified degree of fluidity or rigidity. The fluidity or rigidity of the liposome can be used to control factors such as the stability of the liposome or the rate of release of an entrapped agent. Liposomes having a more rigid lipid bilayer, or a liquid crystalline bilayer, are achieved by incorporation of a relatively rigid lipid. The rigidity of the lipid bilayer correlates with the phase transition temperature of the lipids present in the bilayer. Phase transition temperature is the temperature at which the lipid changes physical state and shifts from an ordered gel phase to a disordered liquid crystalline phase. Several factors affect the phase transition temperature of a lipid including hydrocarbon chain length and degree of unsaturation, charge and headgroup species of the lipid. Lipid having a relatively high phase transition temperature will produce a more rigid bilayer. Other lipid components, such as cholesterol, are also known to contribute to membrane rigidity in lipid bilayer structures. Cholesterol is widely used by those of skill in the art to manipulate the fluidity, elasticity and permeability of the lipid bilayer. It is thought to function by filling in gaps in the lipid bilayer. In contrast, lipid fluidity is achieved by incorporation of a relatively fluid lipid, typically one having a lower phase transition temperature. Phase transition temperatures of many lipids are tabulated in a variety of sources.

In certain aspects, liposomes are made from endogenous phospholipids such as dimyristoyl phosphatidylcholine (DMPC) and dimyristoyl phosphatidylglycerol (DMPG), phosphatidyl serine, phosphatidyl choline, dioleoyphosphatidyl choline [DOPC], cholesterol (CHOL) and cardiolipin.

### III. Methods of Administration and Treatment

Embolism Therapy. Methods of tumor arterial embolism include the injection of an embolus into micro-arteries, causing mechanical blocking and inhibiting tumor growth. In certain aspects, the embolus is a liposome alginate microsphere (LAM) as described herein. In certain aspects, the tumors treated are malignant tumors unsuitable for surgical operations. The tumors can be hepatocellularcarcinoma (HCC), renal cancer, tumors in pelvis and head and neck cancer.

Effectiveness of a microsphere for embolism purposes depends on one or more of microsphere diameter, microsphere degradation rate, and therapeutic agent release rate. The microsphere preparations can block micro-vessels that are supporting the cancer or tumor. The embolism can supply a therapeutic agent that is targeted to the tumor, allowing the therapeutic agent to be targetable and controllable. This kind of drug administration is able to improve drug distribution in vivo and enhance pharmacokinetic features, increase bioavailability of drugs, improving treatment effect, and alleviate toxic or side effects.

1. A method for producing liposome containing alginate microspheres comprising: Atomizing, using an atomizer, a liposome/alginate solution into a curing solution comprising an alginate cross-linker, and isolating liposome containing alginate microsphere having an average diameter of 20 to 80 μm.

2. The method of claim 1, wherein the atomizer is an ultrasonic nozzle.

3. The method of claim 2, wherein the ultrasonic nozzle is a 1 Hz to 100 kHz nozzle.

4. The method of claim 2, wherein the ultrasonic nozzle is a 25 kHz nozzle.

5. The method of claim 1, wherein the atomizer is positioned 1 to 10 cm from the curing solution.

6. The method of claim 1, wherein the curing solution comprises a cation.

7. The method of claim 6, wherein the cation is selected from calcium, strontium, barium, iron, silver, aluminum, magnesium, manganese, copper, and zinc.

8. The method of claim 1, wherein the curing solution comprises CaCl₂.

9. The method of claim 1, wherein the liposome/alginate solution comprises a liposome to alginate ratio of 1:1.

10. The method of claim 1, wherein the liposomes comprise a therapeutic agent or an imaging agent.

11. The method of claim 10, wherein the therapeutic agent is a thermotherapeutic, a chemotherapeutic, or a radiotherapeutic agent.

12. A liposome containing alginate microsphere comprising:
(a) an alginate microsphere having an average diameter of 20 to 80 μm; and
(b) liposomes dispersed in the alginate microsphere, the liposome containing a therapeutic and/or imaging agent.

13. A method for performing embolization therapy on a subject having a tumor comprising injecting the liposome containing alginate microsphere of claim 12 into the tumor vasculature.

14. A thermotherapeutic alginate microsphere comprising a thermotherapeutic agent encapsulated in an alginate microsphere.

15. The microsphere of claim 14, wherein the thermotherpeutic agent is encapsulated in a liposome that is contained in an alginate microsphere.
